(19) United States
(12) Patent Application Publication
(10) Pub. No.: US 2020/0368403 A1
(43) Pub. Date: Nov. 26, 2020

(54) SYSTEMS AND METHODS FOR HIGH-STRENGTH CANISTER RETENTION WITH AUTOMATED, NON-MECHANICAL CANISTER RELEASE FOR USE WITH MEDICAL FLUID COLLECTION SYSTEMS

(71) Applicant: KCI LICENSING, INC., San Antonio, TX (US)

(72) Inventors: Christopher Brian LOCKE, Bournemouth, Dorset (GB); Timothy Mark ROBINSON, Blandford Forum, Dorset (GB)

(73) Assignee: KCI LICENSING, INC., San Antonio, TX (US)

(21) Appl. No.: 16/757,630

(22) PCT Filed: Oct. 22, 2018
(86) PCT No.: PCT/US2018/056834

§ 371 Date: Apr. 20, 2020

Related U.S. Application Data
(60) Provisional application No. 62/575,743, filed on Oct. 23, 2017.

(51) Int. Cl. A61M 1/00; C09J 7/30; C09J 5/00

(52) U.S. Cl. CPC A61M 1/0001; A61M 1/0025; C09J 2205/31; C09J 5/00; C09J 2205/302; C09J 7/30

(57) ABSTRACT

Provided herein is a system and method for attaching and detaching elements of a medical fluid collection system. One aspect provides a high-strength canister retention system with automated, non-mechanical canister release. A canister may have a switchable adhesive that can adhere the canister to a medical therapy unit. When detachment of the canister is desired, the switchable adhesive can be deactivated by exposure to certain light wavelengths, particularly UV light, emitted from a light source disposed in the medical therapy unit. This allows for easy detachment of the canister from the medical therapy unit without the use of mechanical latches.

### CROSS-REFERENCE TO RELATED APPLICATIONS

The application claims the benefit of U.S. Provisional Application No. 62/575,743, filed Oct. 23, 2017, the contents of which are incorporated herein in its entirety.

### BACKGROUND

### 1. Field of Invention

The present application relates generally to the field of medical fluid collection systems, and more specifically to a system and method for high-strength canister retention with automated, non-mechanical canister release for use with medical fluid collection systems.

### 2. Description of Related Art

Canister attachment to medical therapy units has been a challenge for many years. Many attachment systems rely on mechanical latches which require the user to manually operate and to have significant strength to use. Since ease-of-use is so closely aligned with patient compliance and can be directly linked to efficacy of medical treatment outcomes, it is incumbent to have a canister retention system that enables a more diverse and perhaps less skilled group of users to operate the system while maintain the high-strength necessary to retain the canister to a medical therapy unit in a variety of environments. Therefore, a need exists for an electronically switchable canister latch and release technology ideally suited to multiple environments, including environments where the device will be exposed to vibration, shock or impact, such as in ambulatory patient or military applications.

### SUMMARY

To alleviate the existing problems with mechanical latching canister retention systems, the disclosed embodiments describe a high-strength canister retention configuration with automated, non-mechanical canister release for use with medical fluid collection systems. The illustrative embodiments described herein are directed to systems and methods using switchable adhesives for attaching and detaching a canister to a medical therapy unit.

In some embodiments, a canister for use with a medical fluid collection system comprises: a switchable adhesive disposed on a canister surface, the switchable adhesive being configured to adhere the canister to a medical therapy unit. In some embodiments, the switchable adhesive includes: a photosensitive adhesive layer having at least one release agent disposed within the adhesive layer, wherein the at least one release agent is configured to weaken a bond of the adhesive layer to a surface upon exposure to at least one of a plurality of light wavelengths; and a removable blocking layer having at least one blocking agent disposed within the blocking layer, wherein the blocking layer blocks the at least one of a plurality of light wavelengths from exposing the photosensitive adhesive layer. In some embodiments, the plurality of light wavelengths include ultraviolet (UV) light wavelengths. In some embodiments, the plurality of light wavelengths are between 285 nm and 400 nm. In some embodiments, the plurality of light wavelengths are between 320 nm and 370 nm. In some embodiments, the at least one release agent is a photo initiator configured to deactivate the adhesive layer upon exposure to the at least one of a plurality of light wavelengths. In some embodiments, the removable blocking layer is disposed on an outer surface of the photosensitive adhesive layer. In some embodiments, the adhesive layer has a lap shear sufficient to adhere the canister to the medical therapy unit upon activation. In some embodiments, the lap shear of the adhesive layer upon activation is at least 7 kPa. In some embodiments, the adhesive layer has a lap shear sufficiently low enough to enable removal of the canister from the medical therapy unit upon deactivation. In some embodiments, the lap shear of the adhesive layer upon deactivation falls to at least 5 kPa. In some embodiments, the removable blocking layer is integrated into a canister packaging layer and covers the adhesive layer. In some embodiments, the removable blocking layer is a peelable layer that covers the adhesive layer.

In some embodiments, a medical therapy unit for use with a medical fluid collection system comprises: an adhesive layer receiving area disposed on an outside surface of the medical therapy unit configured to allow a passage of at least one of a plurality of light wavelengths through the adhesive layer receiving area; a light source disposed within the medical therapy unit adjacent to the adhesive layer receiving area and configured to emit the at least one of a plurality of light wavelengths; a light diffuser disposed between the light source and the adhesive layer receiving area and configured to focus the at least one of a plurality of light wavelengths to pass through the adhesive layer receiving area; and a light source controller having at least one processor configured to control an ON/OFF state of the light source. In some embodiments, the light source is an ultraviolet (UV) light source and the at least one of a plurality of light wavelengths are UV light wavelengths. In some embodiments, the plurality of light wavelengths are between 285 nm and 400 nm. In some embodiments, the plurality of light wavelengths are between 320 nm and 370 nm.

In some embodiments, the light source controller is configured to receive a light source activation signal and output an ON signal. In some embodiments, the medical therapy unit further comprises a light source driver configured to receive the ON signal from the light source controller and turn the light source to an ON state. In some embodiments, the adhesive layer receiving area is configured to receive a switchable adhesive including a photosensitive adhesive layer having at least one release agent disposed within the adhesive layer, wherein the at least one release agent is configured to weaken a bond of the adhesive layer to a surface upon exposure to the at least one of a plurality of light wavelengths. In some embodiments, the medical therapy unit further comprises an adhesive state sensor configured to detect an adhesion state of a switchable adhesive coupled to the adhesive layer receiving area. In some embodiments, the adhesive state sensor detects the adhesion state of the switchable adhesive by monitoring a conductance of the switchable adhesive. In some embodiments, the adhesive state sensor is an electrical sensor disposed within the light diffuser. In some embodiments, the adhesive state sensor detects whether a non-switchable adhesive is coupled to the adhesive layer receiving area. In some embodiments, the medical therapy unit further comprises a mechanical sensor configured to detect whether a canister is coupled to the medical therapy unit. In some embodiments, the mechanical sensor is disposed within a side wall of the medical therapy unit.

In some embodiments, a medical fluid collection system comprises a medical therapy unit including: an adhesive layer receiving area disposed on an outside surface of the medical therapy unit configured to allow a passage of at least one of a plurality of light wavelengths through the adhesive layer receiving area; a light source disposed within the medical therapy unit adjacent to the adhesive layer receiving area and configured to emit the at least one of a plurality of light wavelengths; a light diffuser disposed between the light source and the adhesive layer receiving area and configured to focus the at least one of a plurality of light wavelengths to pass through the adhesive layer receiving area; and a light source controller having at least one processor configured to control an ON/OFF state of the light source. In some embodiments, the medical fluid collection system further comprises a canister including a switchable adhesive disposed on a canister surface, the switchable adhesive being configured to adhere the canister to the medical therapy unit, the switchable adhesive including a photosensitive adhesive layer having at least one release agent disposed within the adhesive layer, wherein the at least one release agent is configured to weaken a bond of the adhesive layer to a surface upon exposure to the at least one of a plurality of light wavelengths. In some embodiments, the light source is an ultraviolet (UV) light source and the at least one of a plurality of light wavelengths are UV light wavelengths. In some embodiments, the plurality of light wavelengths are between 285 nm and 400 nm. In some embodiments, the plurality of light wavelengths are between 320 nm and 370 nm. In some embodiments, the adhesive layer receiving area is configured to receive the switchable adhesive and shield the switchable adhesive from exposure to ambient light. In some embodiments, the canister is configured to detach from the medical therapy unit upon exposure of the switchable adhesive to the at least one of a plurality of light wavelengths emitted from the light source. In some embodiments, upon detachment from the medical therapy device, the canister is configured to be pushed away from the medical therapy device by a sealing bellows and retained by a retaining element disposed on the medical therapy device. In some embodiments, the medical therapy device further comprises at least one protrusion disposed on an outside surface of the medical therapy device. In some embodiments, the canister further comprises at least one recess disposed on an outside surface of the medical therapy device. In some embodiments, the at least one recess is configured to receive the at least one protrusion to couple the canister to the medical therapy device. In some embodiments, the at least one recess comprises at least one receptacle disposed within the at least one recess, the at least one receptacle containing a plurality of adhesive precursors. In some embodiments, the at least protrusion is configured to puncture the at least one receptacle upon insertion into the at least one recess and enable the plurality of adhesive precursors to mix and form an adhesive to adhere the canister to the medical therapy device. In some embodiments, the formed adhesive is a switchable adhesive configured to be deactivated upon exposure to the at least one of a plurality of light wavelengths emitted from the light source.

In some embodiments, a method of operating a medical fluid collection system comprises: adhering a canister to a medical therapy system, the adhering including: removing a removable blocking layer from a photosensitive adhesive layer disposed on an outside surface of the canister; and attaching the adhesive layer to an adhesive layer receiving area disposed on an outside surface of the medical therapy unit; and detaching the canister from the medical therapy device, the detaching including: activating a light source disposed within the medical therapy unit adjacent to the adhesive layer receiving area and configured to emit at least one of a plurality of light wavelengths; exposing the photosensitive adhesive layer to at least one of a plurality of light wavelengths, wherein the photosensitive adhesive layer is deactivated upon exposure to the at least one of a plurality of light wavelengths; and retaining the canister in a retaining element disposed on the outside of the medical therapy device after detachment.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-1D are cross-sectional views of a light deactivated (switchable) adhesive system in accordance with an illustrative embodiment of the present disclosure.

FIG. 2 is a diagram of an exemplary medical fluid collection system using a non-mechanical canister retention system in accordance with another illustrative embodiment of the present disclosure.

FIG. 3 is a flowchart illustrating a process for using the non-mechanical canister retention system shown in FIG. 2 in accordance with an illustrative embodiment of the present disclosure.

### DETAILED DESCRIPTION

In the following detailed description of the preferred embodiments, reference is made to the accompanying drawings that form a part hereof, and in which is shown by way of illustration specific preferred embodiments in which the invention can be practiced. These embodiments are described in sufficient detail to enable those skilled in the art to practice the invention, and it is understood that other embodiments can be utilized and that logical structural, mechanical, electrical, and chemical changes can be made without departing from the spirit or scope of the invention. To avoid detail not necessary to enable those skilled in the art to practice the invention, the description can omit certain information known to those skilled in the art. It is understood that reference to a feature by numeric designation does not necessarily refer only to any particular embodiment depicted in a drawing. The following detailed description is, therefore, not to be taken in a limiting sense, and the scope of the present invention is defined only by the appended claims.

The following definitions are provided to better define the present invention and to guide those of ordinary skill in the art in the practice of the present invention. Unless otherwise noted, terms are to be understood according to conventional usage by those of ordinary skill in the relevant art.

The medical therapy device and canister described herein is useful in negative pressure wound treatment (NPWT) devices, although the disclosed embodiments can work with a range of canisters and products and is ideally suited to homecare therapy systems. The disclosed embodiments enable mechanical latch points and/or mechanisms on the canister to be removed as well as corresponding latch mechanisms on the medical therapy unit. This alleviates the need for a user to apply a certain manual force to the system to detach the canister from the medical therapy device when the canister needs to be replaced. As a result, users of different physical capabilities can easily and safely operate the disclosed systems.

In the disclosed embodiments, a light source is integrated into a canister-side housing of a medical therapy unit. A portion of the outside surface of the canister is supplied with an area of light-deactivated (switchable) adhesive. The adhesive is protected from environmental light by a blocking layer integrated within the canister packaging or onto a surface of the adhesive and which faces the canister wall. As the canister is removed from the packaging, the blocking layer which is attached to the packaging is removed from the canister, thereby exposing the adhesive material. In some embodiments, the adhesive is designed such that the adhesive will not switch to an inert state in ambient light but is tuned to a frequency at an upper end of the ultraviolet (UV) spectrum. When the user attaches a canister to the medical therapy unit, they first remove the canister from the packaging as discussed above and “stick” the canister to the side of the medical therapy unit via the adhesive. The design of both is such that the adhesive is disposed in an area of the medical therapy housing that lines up with an area of a triggering light diffuser disposed inside the medical therapy housing. In this way, a robust bond over a large area is obtained rapidly by this high-tack adhesive and can be easily removed by triggering a deactivation of the adhesive by activating the light source.

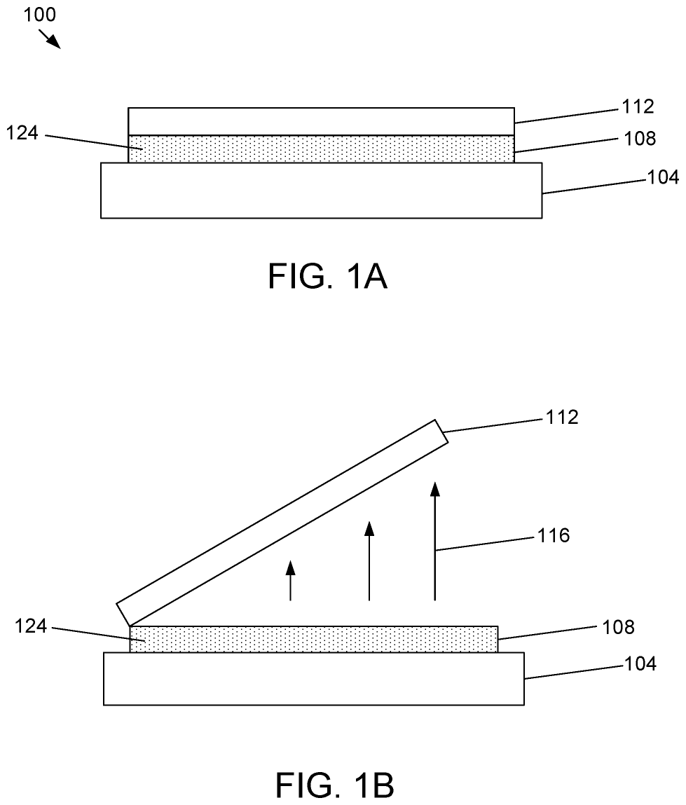

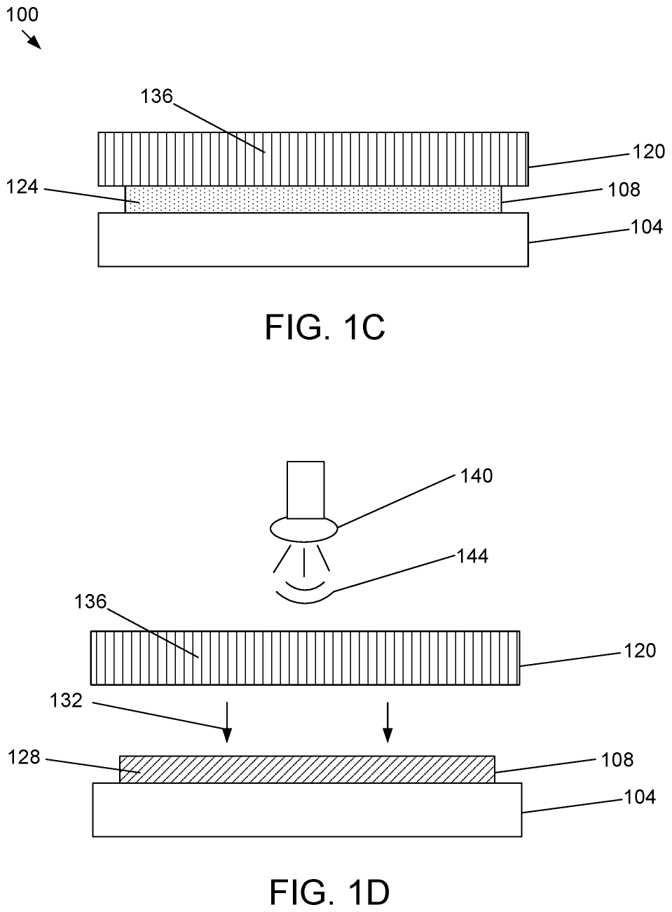

Referring more specifically to the drawings, FIGS. 1A-1D show cross-sectional views of a light deactivated (switchable) adhesive system 100 in accordance with an illustrative embodiment of the present disclosure. As shown in FIG. 1A, the system 100 comprises a surface 104 (e.g., a surface layer), a photosensitive adhesive layer 108, and a blocking layer 112. In the embodiment shown, adhesive layer 108 is affixed to the surface 104 on one side of the adhesive layer and coupled to blocking layer 112 on a side opposite the side affixed to surface 104. In the embodiment shown, surface 104 is an outside surface of a canister. In the embodiment shown, the adhesive layer 108 may cover any portion of the surface 104 as may be required to securely affix the canister to the medical therapy unit. The adhesive layer 108 can comprise any material, in single or multiple layers, capable of adhering to surface 104.

In the embodiment shown, prior to attaching the canister to the medical therapy unit, the adhesive layer 108 is covered by blocking layer 112. Blocking layer 112 is a removable layer that covers the adhesive surface of the adhesive layer 108 until the adhesive layer is desired to be adhered to another surface such as a housing surface of the medical therapy unit. As shown in FIG. 1B, the blocking layer 112 can be peeled off or otherwise removed with a force (represented by arrows 116) to expose an adhesive surface of adhesive layer 108 before applying the adhesive surface to a surface 120 (as shown in FIG. 1C). The adhesive layer 108 can comprise one or more materials including, but not limited to, polyurethane, acrylic (e.g., cyanoacrylate), hydrogel, silicon or silicone based material, natural rubber, synthetic rubber, styrene block copolymers, polyvinyl ethers, poly(meth)acrylates, polyolefins, hydrocolloid (e.g., a rubber based hydrocolloid), or a combination thereof. In some embodiments, the adhesive layer 108 comprises a polymer or co-polymer. For example, the adhesive layer 108 can comprise a co-polymer of polyurethane and silicone or various acrylic co-polymers.

In the embodiment shown in FIG. 1A, the adhesive layer 108 may include at least one release agent 124 comprising a release material. In the embodiment shown, adhesive layer 108 has a plurality of release agents 124 (represented by dots). The release agent 124 can physically or chemically affect adhesion characteristics of adhesive layer 108. A release agent 124 can comprise a variety of molecular compositions depending on the particular embodiment being implemented, including but not limited to a photopolymer, an oil particle, a gas particle, a solvent, a lipid, and/or one or more microstructures. Release agents 124 can be present in an inert or inactive form in, on, or near an adhesive layer 108. For example, a release agent 124 can be mixed with the adhesive; on the surface of the adhesive with a random or patterned coverage; coupled to the drape with a random or patterned coverage; or contained within a microstructure located in these or other locations. Upon release or activation, release agents 124 can migrate within the adhesive layer 108 or along an interface between an adhesive layer 108 and surface 120 to facilitate the removal of a canister (e.g., surface 104) from the medical therapy unit (e.g., surface 120 of FIG. 1C). In the embodiment shown, the release agent 124 is configured to transition from an unreleased state (shown in FIG. 1A) to a release state 128 (represented by diagonal lines in FIG. 1D) to weaken a bond of the adhesive layer 108 to surface 120 upon exposure to an external stimulus. Various external stimulus can be employed depending on the particular embodiment being implemented. Non-limiting examples of the external stimulus include electromagnetic (e.g., UV, visible, or infrared light), magnetic, sound, pH, pressure (e.g., positive atmospheric pressure, negative atmospheric pressure, shear force, direct force), thermal, moisture, or a substance. The external stimulus can also be a substance, compound, liquid, or gas capable of reacting with a release agent 124 in adhesive layer 108 such that the release agent 124 transitions from an unreleased state to a released state. In the embodiment shown, the external stimulus is one or more of a plurality of light wavelengths. The weakened bond that occurs as a result of the release of release agent 124 allows a user of the system 100 to apply a force on surface 104, such as a force indicated by arrow 132, to remove adhesive layer 108 from tissue 120.

Referring more specifically to FIG. 1A, in the embodiment shown, release agents 124 are inertly dispersed within adhesive layer 108 and can be located anywhere within adhesive layer 108, as well as any of the outer surfaces of adhesive layer 108, such as an interface between adhesive layer 108 and blocking layer 112. In some embodiments, a separate film layer (not shown in FIG. 1A), can separate release agents 124 from adhesive layer 108. In these embodiments, the presence of an external stimulus can weaken, break-down, or increase the permeability of the separate film layer such that release agents 124 are allowed to migrate into adhesive layer 108 to facilitate the removal of adhesive layer 108 from surface 120.

As shown in FIG. 1B, blocking layer 112 is removed from the adhesive surface of adhesive layer 108 via force 116. This exposes the adhesive surface of adhesive layer 108 and enables it to adhere to surface 120, as shown in FIG. 1C. In some embodiments, blocking layer 112 is disposed in a canister packaging and may be removed by removing the canister packaging from the canister. In other embodiments, blocking layer 112 is a removable label disposed on the adhesive surface of adhesive layer 108 that can be peeled off or otherwise removed from the surface of adhesive layer 108. In the embodiment shown, surface 120 has various apertures or passageways (denoted by lines 136) that allow certain wavelengths of light to pass through surface 120. As shown in FIG. 1D, release agents 124 may be released in the presence of external stimulus such that release agents 124 are allowed to migrate within adhesive layer 108 and the interface between adhesive layer 108 and tissue 120. In the embodiment shown, a UV light source 140 emits a plurality of light wavelengths 144 that pass through passageways 136 and expose adhesive layer 108 to the plurality of light wavelengths 144. In some embodiments, exposure to the plurality of light wavelengths 144 can cause microstructures containing release agents 124 to rupture or tear, thereby releasing release agents 124 from the interior of the microstructures. These released release agents 124 can then be interspersed into adhesive layer 108 and the interface between adhesive layer 108 and surface 120, thereby weakening the bond between adhesive layer 108 and surface 120 and facilitating the removal of adhesive layer 108 from surface 120. As the plurality of light wavelengths 144 reach adhesive 108, release agents 124 may transition from an unreleased state (as shown in FIG. 1A) to a released state 128 (as shown in FIG. 1D) as they are exposed to the plurality of light wavelengths 144. In the embodiment shown, the plurality of light wavelengths 144 are UV wavelengths. In some embodiments, the UV wavelengths may be within a range of 285 nm-400 nm, although it may be preferable to have the UV wavelengths be UVA wavelengths within a range of 320 nm-370 nm.

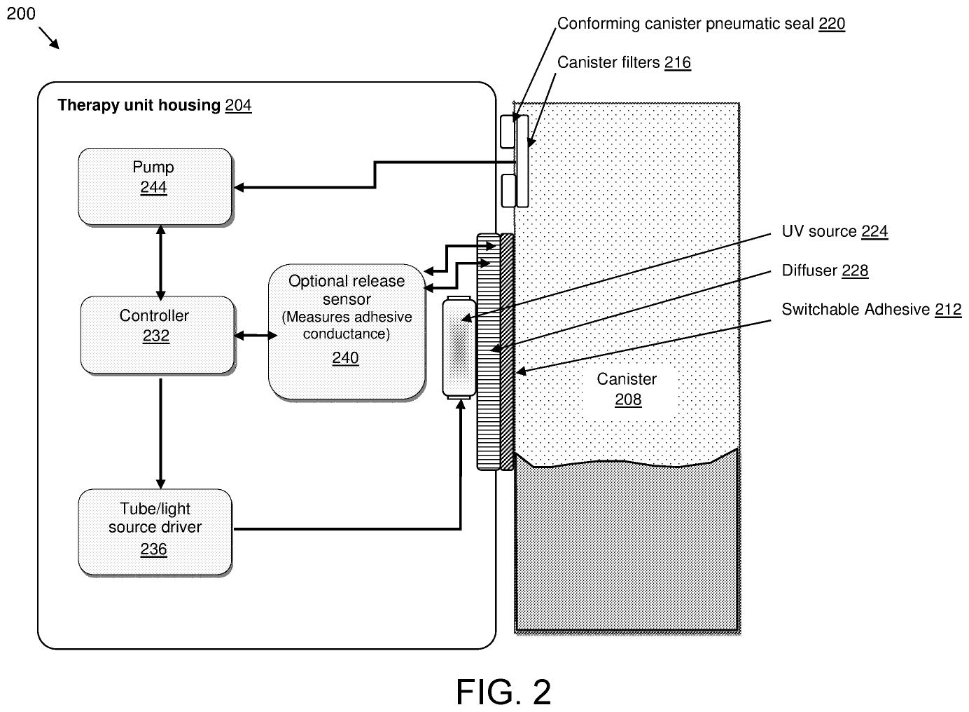

FIG. 2 is a diagram of an exemplary medical fluid collection system 200 using a non-mechanical canister retention system in accordance with another illustrative embodiment of the present disclosure. In the embodiment shown, system 200 includes a medical therapy unit 204 and a canister 208. In the embodiment shown, medical therapy unit 204 may include a housing having a plurality of outside surfaces that surround multiple components disposed within the housing. Canister 208 can be any canister useful for medical fluid collection or any other medical use and can be filled with any fluid, either liquid or gaseous. In the embodiment shown, canister 208 has a switchable adhesive 212 disposed on a portion of the canister's outer surface. Switchable adhesive 212 can comprise a light deactivated adhesive layer as discussed previously. For example, the canister 208 may include or correspond to the surface 104, and the switchable adhesive may include or correspond to the adhesive layer 108. In the embodiment shown, canister 208 can have an interface that includes canister filters 216 for attaching to medical therapy device 204 via a conforming canister pneumatic seal 220. This interface enables fluid to pass between medical therapy device 204 and canister 208 in a single direction or in a bidirectional manner depending upon the specific application of medical therapy device 204 and canister 208.

In the embodiment shown, medical therapy unit 204 includes various components disposed inside a housing, including a light source 224, a light source diffuser 228, and a light source controller 232. The light source 224 may include or correspond to the UV light source 140, and the light source diffuser 228 may include or correspond to the surface 120. In some embodiments, medical therapy unit 204 also includes a light source driver 236 and one or more optional release sensors 240. In the embodiment shown, medical therapy unit 204 includes a pump 244 for dispensing and/or receiving fluid to and/or from canister 208 via the interface of canister filters 216 and canister pneumatic seal 220.

In the embodiment shown, canister 208 is attached to an outside surface or housing of medical therapy unit 204 via switchable adhesive 212. As discussed previously, blocking layer 112 can be removed from switchable adhesive 212 to enable switchable adhesive to firmly adhere to medical therapy unit 204. In the embodiment shown, switchable adhesive 212 is adhered to a specific area (i.e., an adhesive receiving area) of the surface or housing of medical therapy unit 204 specifically designed to receive the switchable adhesive 212. This adhesive receiving area can include the various apertures or passageways (denoted by lines 136 in FIG. 1C) discussed previously that allow certain wavelengths of light to pass through the surface or housing of medical therapy unit 204 contained within the adhesive receiving area. In the embodiment shown, the adhesive receiving area can completely cover the surface of switchable adhesive 212 to prevent exposure of the surface of switchable adhesive 212 to ambient light that could possibly prematurely deactivate switchable adhesive 212.

In the embodiment shown, light diffuser 228 is disposed adjacent to the adhesive receiving area on an inside surface of medical therapy unit 204. In the embodiment shown, light diffuser 228 is disposed between light source 224 and the adhesive receiving area. In the embodiment shown, light source 224 has both ON and OFF switching states controlled by controller 232. In a default state that enables switchable adhesive 212 to be adhered to medical therapy unit 204, light source 224 is in an OFF switching state. In the OFF switching state, light source 224 does not emit light wavelengths. In the embodiment shown, light source 224 is a UV light source configured to emit UV light wavelengths. However, in some embodiments, other types of light (e.g., visible, infrared) can be used.

In some embodiments, controller 232 can comprise at least one processor and can be controlled by a user of system 200. For example, a user can send inputs to controller 232 via one or more manual controls such as switches or buttons. These manual controls can direct controller 232 to switch between the ON and OFF switching states of light source 224. In other embodiments, controller 232 can be controlled automatically, such as via a system of sensors and computer-readable media having executable instructions for execution by the at least one processor of the controller 232. In these embodiments, one or more sensors can detect a state of canister 208 to determine a desired time for detachment of the canister 208 from medical therapy unit 204. These sensors can include fluid level sensors, fluid pressure sensors, or other suitable sensors. In the embodiment shown, controller 232 can control and receive feedback from pump 244 to control the transfer of fluid between medical therapy unit 204 and canister 208.

In the embodiment shown, controller 232 controls light source driver 236 that switches light source 224 between ON and OFF states. When the canister 208 is desired to be detached from medical therapy unit 204, controller 232 sends instructions to light source driver 236 to turn light source 224 to an ON state. During an ON state, light source 224 emits a plurality of light wavelengths toward and through light source diffuser 228. Light source diffuser 228 focuses the emitted plurality of light wavelengths to pass through the adhesive receiving area of the surface or housing of the medical therapy unit 204. Switchable adhesive 212 is exposed to the plurality of light wavelengths that pass through the adhesive receiving area and switches to a deactivated or inert state as discussed previously. This enables canister 208 to be detached from medical therapy unit 204. In some embodiments, one or more retaining elements (e.g., receptacles, rails, etc.) can be disposed on the outside of medical therapy unit 204 to retain the detached canister 208 in secure position until the canister can be properly removed and/or disposed of. In the embodiment shown, one or more optional release sensors 240 can detect the state of the switchable adhesive 212 (i.e., activated or deactivated state) and input this information to controller 232. In other implementations, light source 224 may be configured to be activated (e.g., turned on) and/or deactivated (e.g., turned off) based on operation of a switch, such as a manually operated switch, incorporated in housing 204.

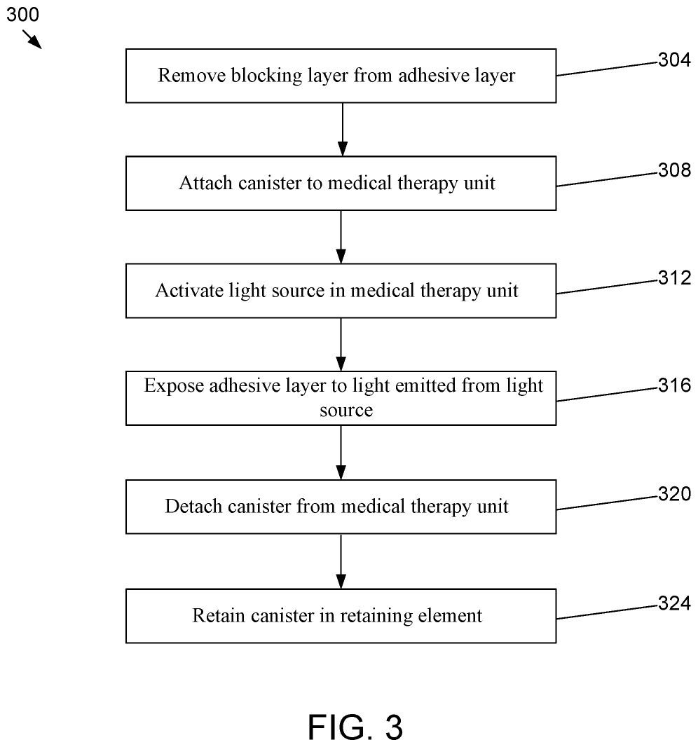

FIG. 3 is a flowchart illustrating a process 300 for using the non-mechanical canister retention system shown in FIG. 2 in accordance with an illustrative embodiment of the present disclosure. The process illustrated in FIG. 3 can be implemented by a user of system 200. The process begins by removing blocking layer 112 from adhesive layer 108 (step 304). In this step, the adhesive surface of adhesive layer 108 is exposed and can bind to the surface 120 of medical therapy unit 204. In step 308, canister 208 can be attached to medical therapy unit 204 by applying switchable adhesive 212 to the adhesive receiving area of the medical therapy unit 204. When it is desired to detach canister 208 from medical therapy unit 204, light source 224 can be activated by controller 232 at step 312. As discussed previously, this can be done either manually by a user actuating a switch or may be done automatically based on feedback received by controller 232 (e.g., signals from sensors and/or instructions from computer-readable media). Upon activation of light source 224, the switchable adhesive 212 is exposed to the plurality of light wavelengths emitted from light source 224 at step 316. The plurality of light wavelengths can stimulate release agents 124 to deactivate the adhesive and enable detachment of the canister from the medical therapy unit 204 at step 320. In some embodiments, the positioning and/or weight of the canister can cause the canister detachment. In other embodiments, one or more mechanical elements can contact (e.g., push) the canister to promote detachment. At step 324, the detached canister can be retained in a retaining element to maintain the detached canister in a secure position. For example, the canister can be pushed away from medical therapy unit 204 near its top end by a sealing bellows disposed near the canister pneumatic seal 220 and is also retained when released by mechanical features disposed on the surface or housing of medical therapy unit 204. The switched canister releases due to the weight of the full canister and tips back into the retained but released position awaiting replacement. Any attempt made to re-attach the canister will fail because the switchable adhesive is inert or deactivated. In some embodiments, the switchable adhesive contains photo initiators (PI's) that are sensitive to the UVA wavelength range of 285-400 nm and, more specifically, 320-370 nm.

In some embodiments, the switchable adhesive can be a double-sided adhesive strip 2 cm wide by 5 cm long, 250 microns thick. The canister can of any size used with medical systems, such a 500 cc or 1L canisters. A full 500 cc canister usually contains about 500 g of fluid (the equivalent of 5N) so that, when the adhesive is switched off (after exposure to UVA), the lap shear of the adhesive should fall to at least 5 kPa ([5N/0.001 m2]. This calculation ignores the mass of the canister material and any attached tubing, which can increase the ease of canister release upon light activation. To permit the 500 cc canister to remain securely attached to the therapy device while the adhesive is active, the adhesive strip has a lap shear of at least 7 kPa (the mass of 500 cc of fluid, plus about 200 g of canister and tubing material). For a one liter canister, the adhesive can be expected to have about double the lap shear values (e.g., 14 kPa) of the 500 cc canister assembly. Alternatively, the adhesive strip can have double the area of an adhesive strip used with a 500 cc canister.

In some embodiments, the system 200 has an electrical sensor 240 configured to receive signals from diffuser 228 or integrated within diffuser 228 which monitors the conductance of the switchable adhesive 212 to determine its presence and whether the adhesive has been switched. In these embodiments, sensor 240 may also be used to determine if a counterfeit or deactivated adhesive strip has been applied, thus preventing multiple uses. In some embodiments, other counterfeiting detection aids may be incorporated into the adhesive, such as fluoresces, which may be detected with a suitable sensor on or in the medical therapy unit 204. In some embodiments, system 200 can have one or more mechanical or other sensors that may be integrated with a side wall of the medical therapy unit 204 to detect (using the Hall effect or other suitable means) canister engagement and whether the canister has been disengaged from the medical therapy unit 204.

In some embodiments, to prevent uncontrolled multiple use of the canister by simply applying other types of double-sided adhesives, one or more pins or rods may be used on the medical therapy device to engage with one or more matching holes or recesses in the canister. This configuration does not permit any useful adjacent flat-to-flat surface contact, thus preventing any non-switchable adhesives from being used. In these embodiments, one or more domes or other shapes can be disposed within the recesses and can be filled with reactive adhesive precursors (e.g., in two-part systems) that, when mixed, form a strong adhesive to fix the therapy device pins/rods in place within the recesses. The domes can be fractured by the entry of the therapy device rods to release and mix the adhesive precursors. In these embodiments, the adhesive would also contain a UVA PI system that would enable the bond to the therapy device rod to be deactivated and detached with little applied force.

The flowcharts and block diagrams in the different depicted embodiments illustrate the architecture, functionality, and operation of some possible implementations of the apparatus and methods. In some alternative implementations, the function or functions noted in the block can occur out of the order noted in the figures. For example, in some cases, two blocks shown in succession can be executed substantially concurrently, or the blocks can sometimes be executed in the reverse order, depending upon the functionality involved.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the disclosed methods, devices, and systems are not intended to be limited to the particular forms disclosed. Rather, they include all modifications and alternatives falling within the scope of the claims, and embodiments other than those shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A canister for use with a medical fluid collection system, the canister comprising:
a switchable adhesive disposed on a canister surface, the switchable adhesive configured to adhere the canister to a medical therapy unit, the switchable adhesive including a photosensitive adhesive layer having at least one release agent disposed within the photosensitive adhesive layer, wherein the at least one release agent is configured to weaken a bond of the photosensitive adhesive layer to a surface upon exposure to at least one of a plurality of light wavelengths; and
a removable blocking layer having at least one blocking agent disposed within the removable blocking layer, wherein the removable blocking layer is configured to block the at least one of the plurality of light wavelengths from exposing the photosensitive adhesive layer.

2. The canister of claim 1, wherein:
the plurality of light wavelengths include ultraviolet (UV) light wavelengths; and
the plurality of light wavelengths are between 285 nm and 400 nm.

3-4. (canceled)

5. The canister of claim 1, wherein the at least one release agent is a photo initiator configured to deactivate the photosensitive adhesive layer upon exposure to the at least one of the plurality of light wavelengths.

6. The canister of claim 1, wherein the removable blocking layer is disposed on an outer surface of the photosensitive adhesive layer.

7. The canister of claim 1, wherein:
the photosensitive adhesive layer has a lap shear sufficient to adhere the canister to the medical therapy unit upon activation;
the lap shear of the photosensitive adhesive layer upon activation is at least 7 kPa;
the lap shear of the photosensitive adhesive layer upon deactivation is sufficiently low to enable removal of the canister from the medical therapy unit; and
the lap shear of the photosensitive adhesive layer upon deactivation falls to at least 5 kPa.

8-10. (canceled)

11. The canister of claim 1, wherein the removable blocking layer is integrated into a canister packaging layer and covers the photosensitive adhesive layer.

12. The canister of claim 1, wherein the removable blocking layer comprises a peelable layer that covers the photosensitive adhesive layer.

13. A medical fluid collection system comprising:
a medical therapy unit comprising:
an adhesive layer receiving area disposed on an outside surface of the medical therapy unit configured to allow a passage of at least one of a plurality of light wavelengths through the adhesive layer receiving area;
a light source disposed within the medical therapy unit adjacent to the adhesive layer receiving area and configured to emit the at least one of the plurality of light wavelengths;
a light diffuser disposed between the light source and the adhesive layer receiving area and configured to focus the at least one of the plurality of light wavelengths to pass through the adhesive layer receiving area; and
a light source controller having at least one processor configured to control an ON/OFF state of the light source.

14. The medical fluid collection system of claim 13, further comprising:
a canister comprising a switchable adhesive disposed on a canister surface, the switchable adhesive being configured to adhere the canister to the medical therapy unit, the switchable adhesive including a photosensitive adhesive layer having at least one release agent disposed within the photosensitive adhesive layer, wherein the at least one release agent is configured to weaken a bond of the photosensitive adhesive layer to a surface upon exposure to the at least one of the plurality of light wavelengths; and
wherein the light source is an ultraviolet (UV) light source and the at least one of the plurality of light wavelengths are UV light wavelengths.

15. The medical fluid collection system of claim 14, wherein:
the canister is configured to detach from the medical therapy unit upon exposure of the switchable adhesive to the at least one of the plurality of light wavelengths emitted from the light source; and
upon detachment from the medical therapy unit, the canister is configured to be pushed away from the medical therapy unit by a sealing bellows and retained by a retaining element disposed on the medical therapy unit.

16. The medical fluid collection system of claim 14, wherein:
the medical therapy unit further comprises at least one protrusion disposed on an outer surface of the medical therapy unit; and
the canister further comprises at least one recess disposed on an outer surface of the canister the at least one recess configured to receive the at least one protrusion to couple the canister to the medical therapy unit.

17. The medical fluid collection system of claim 13, wherein:
the light source controller is configured to receive a light source activation signal and output an ON signal; and
the medical therapy unit further comprises a light source driver configured to receive the ON signal from the light source controller and turn the light source to an ON state.

18. (canceled)

19. The medical fluid collection system of claim 13, wherein:
the adhesive layer receiving area is configured to receive a switchable adhesive including a photosensitive adhesive layer having at least one release agent disposed within the photosensitive adhesive layer; and
the at least one release agent is configured to weaken a bond of the photosensitive adhesive layer to a surface upon exposure to the at least one of the plurality of light wavelengths.

20. The medical fluid collection system of claim 13, wherein:
the medical therapy unit further comprises an adhesive state sensor configured to detect an adhesion state of a switchable adhesive coupled to the adhesive layer receiving area by monitoring a conductance of the switchable adhesive;
the adhesive state sensor is further configured to detect whether a non-switchable adhesive is coupled to the adhesive layer receiving area; and
the adhesive state sensor comprises an electrical sensor disposed within the light diffuser.

21-23. (canceled)

24. The medical fluid collection system of claim 13, wherein:
the medical therapy unit further comprises a mechanical sensor configured to detect whether a canister is coupled to the medical therapy unit; and
the mechanical sensor is coupled to a side wall of the medical therapy unit.

25-38. (canceled)

39. A method of operating a medical fluid collection system, the method comprising:
adhering a canister to a medical therapy unit, the adhering including:
removing a removable blocking layer from a photosensitive adhesive layer disposed on an outside surface of the canister; and
attaching the photosensitive adhesive layer to an adhesive layer receiving area disposed on an outside surface of the medical therapy unit; and
detaching the canister from the medical therapy unit, the detaching including:
activating a light source disposed within the medical therapy unit adjacent to the adhesive layer receiving area and configured to emit at least one of a plurality of light wavelengths;
exposing the photosensitive adhesive layer to at least one of the plurality of light wavelengths, wherein the photosensitive adhesive layer is deactivated upon exposure to the at least one of the plurality of light wavelengths; and
retaining the canister in a retaining element disposed on the outside of the medical therapy unit after detachment.

40. The method of claim 39, wherein activating the light source disposed within the medical therapy unit comprises automatically activing the light source based on feedback received from a controller of the medical therapy unit.

41. The method of claim 39, wherein the at least one of the plurality of light wavelengths is configured to stimulate release agents embedded within the photosensitive adhesive layer to deactivate the photosensitive adhesive layer.

42. The method of claim 39, wherein retaining the canister in the retaining element comprises pushing the canister away from the medical therapy unit via a sealing bellows positioned proximate to a pneumatic seal of the canister.

43. The method of claim 39, further comprising preventing multiple uses of the canister by engaging a pin of the medical therapy unit with a recess of the canister to prevent contact between a surface of the canister and a surface of the medical therapy unit.
